(19) United States
(12) Patent Application Publication
(10) Pub. No.: US 2012/0012127 A1
(43) Pub. Date: Jan. 19, 2012

(54) COSMETIC COMPOSITIONS CONTAINING ARGININE AND A PRESERVATIVE SYSTEM CONTAINING AN ALCOHOL

(75) Inventors: Maria Pia Rossi, Hillsboro, OR (US); Bruno Bavouzet, Gentilly (FR); Lilavati Patel, Edison, NJ (US)

(73) Assignee: L'OREAL, Paris (FR)

(21) Appl. No.: 13/078,418

(22) Filed: Apr. 1, 2011

Related U.S. Application Data
(60) Provisional application No. 61/320,028, filed on Apr. 1, 2010.

(51) Int. Cl. A61K 8/44; A61Q 1/10

(52) U.S. Cl. 132/202; 424/70.7

(57) ABSTRACT

Disclosed are cosmetic compositions containing a arginine, and a preservative system comprising from about 0.5% to about 15% of an alcohol, by weight, based on the total weight of the cosmetic composition. Also disclosed are methods for making the cosmetic composition and applying it to keratinous tissue such as skin in order to enhance its appearance.

### CROSS-REFERENCE TO RELATED APPLICATIONS

This application claims the benefit of the filing date of U.S. Provisional Patent Application No. 61/320,028, filed Apr. 1, 2010, the disclosure of which is hereby incorporated herein by reference.

### BACKGROUND OF THE INVENTION

The present invention generally relates to a novel cosmetic composition including mascara, and a method of use, including a method of making-up keratinous tissues including eyelashes. More particularly, the present invention relates to a mascara composition which has good physical properties and long wear and is resistant to microbiological contamination for extended periods of time, which can be achieved through the use of a combination of arginine and a preservative system containing an alcohol.

Preservatives are widely used in the cosmetics industry to prevent microbiological contamination of a cosmetic product during storage. Conventional cosmetic preservatives can be ineffective preventing microbiological contamination in cosmetic compositions containing ingredients, such as amino acids, that are particularly susceptible to contamination. For example, at high pH parabens can become hydrolyzed and thus rendered ineffective as a preservative in a cosmetic composition. In addition, too high a concentration of parabens (e.g., methylparaben) in an amino acid-containing composition can disrupt and thus destabilize an emulsion. Further, the pH adjuster or neutralizer triethanolamine tends to degrade into nitrosamine byproducts, which are feared to be carcinogenic.

Therefore, it is desirable to provide cosmetic compositions, such as mascara, that are resistant to microbiological contamination, which do not suffer from the drawbacks of conventional preservatives and neutralizers.

### SUMMARY OF THE INVENTION

A first aspect of the present invention is directed to a cosmetic composition, including mascara, containing arginine and a preservative system comprising from about 0.5% to about 15% of an alcohol, by weight, based on the total weight of the cosmetic composition. Typically, the compositions further contain at least one additional cosmetically acceptable ingredient.

A second aspect of the present invention is directed to a method of preparing a cosmetic composition, including mascara, by mixing together arginine and a preservative system comprising from about 0.5% to about 15% of an alcohol, by weight, based on the total weight of the cosmetic composition.

A third aspect of the present invention is directed to a method of making up or enhancing the appearance of a keratinous tissue by applying to the keratinous tissue a cosmetic composition, including mascara, containing arginine and a preservative system comprising from about 0.5% to about 15% of an alcohol, by weight, based on the total weight of the cosmetic composition.

The inventive compositions may be formulated for application to keratinous tissue, including hair, skin and nails, and for which the presence of arginine is desirable from any cosmetic standpoint. The invention is particularly useful in formulating cosmetic compositions that contain one or more ingredients that require or are preferably formulated with a neutralizer, e.g., various acrylate-based thickeners such as Carbopols, and hydrophobically modified alkali-soluble emulsions (HASE) (commercially available from Rohm & Haas under the tradename Aculyn) and C₁₂-C₁₆ fatty acids. The latter ingredients have been proven useful in mascara compositions in that they provide the desirable properties of creamy texture and a volumizing effect. In the course of developing their invention, and particularly in the embodiments directed to mascara compositions, Applicants surprisingly and unexpectedly discovered that by using arginine instead of triethanolamine, the inventive preservative systems not only provide excellent resistance to microbial contamination, but also do not disrupt or destabilize the emulsion, and maintain excellent color dispersion. It was also discovered that other cosmetically acceptable preservatives, e.g., parabens and benzoates, did not provide both of these attributes. Thus, the present invention provides, in some embodiments, cosmetic compositions containing arginine, as a pH regulator for example, emulsions containing fatty acid emulsifiers such as stearic acid, that are both microbiologically stable and free of other preservatives, such as parabens, and also free of triethanolamine as a neutralizer.

### DETAILED DESCRIPTION

Other than in the operating examples, or where otherwise indicated, all numbers expressing quantities of ingredients and/or reaction conditions are to be understood as being modified in all instances by the term “about,” meaning within 10% to 15% of the indicated number.

“Keratinous tissue”, as used herein, includes but is not limited to, skin, hair and nails.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 100° C. “Non-volatile”, as used herein, means having a flash point of greater than about 100° C.

As used herein, the expressions “at least one”, “a”, and “an” mean one or more and thus include individual components as well as mixtures/combinations.

“Long wear” compositions as used herein, refer to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions.

### Arginine

According to the present invention, compositions comprising arginine are provided. Arginine is a semi-essential amino acid. Typically, arginine is found in the L-form (commercially available from Ajinomoto), which is bioavailable to mammals. In embodiments of the present invention, arginine is a pH regulator of, for example, stearic acid in cosmetic emulsions. In an embodiment of the present invention the arginine is a natural or synthetic L-arginine. Arginine is present in the inventive compositions in amounts generally ranging from about 0.1% to about 5%, in some embodiments from about 0.2% to about 2%, in some embodiments from about 0.5% to about 1.5%, by weight, based on the total weight of the cosmetic composition. In embodiments of the present invention arginine is present in an amount of about 1.1%, by weight, based on the total weight of the composition.

### Preservative System

According to the present invention, compositions comprising a preservative system containing alcohol are provided. The preservative system of the present invention generally contains from about 0.5% to about 15% of an alcohol, and in certain embodiments from about 1% to about 7%, by weight, based on the total weight of the cosmetic composition. Representative examples of suitable alcohols include chlorphenesin and short-chain monoalcohols, for example of C₁-C₄, for instance ethanol or isopropanol; diols or polyols, for instance ethylene glycol, 1,2-pentanediol, 1,2-propylene glycol, phenoxyethanol, caprylyl glycol, hexylene glycol, diethylene glycol, dipropylene glycol, butylene glycol, 2-ethoxyethanol, diethylene glycol monomethyl ether, triethylene glycol monomethyl ether and sorbitol, and combinations of two or more thereof.

In some embodiments, the preservative system contains a glycol, e.g., pentylene glycol or caprylyl glycol. Amounts of the glycol typically range from about 0.5% to about 2.5%, and in some embodiments from about 0.5 to about 1.5%, and in yet other embodiments, from about 0.5% to about 1.0%, by weight, based on the total weight of the composition.

In some embodiments, the preservative system contains ethanol. Amounts of ethanol typically range from about 1.5% to about 15%, and in some embodiments from about 1.5% to about 10%, and in other embodiments from about 2.0% to about 6.0%, and in yet other embodiments, from about 2.5% to about 3.5%, by weight, based on the total weight of the composition.

In preferred embodiments, the preservative system contains a combination of a glycol, e.g., pentylene glycol or caprylyl glycol, and ethanol.

In other embodiments, the preservative system further contains phenoxyethanol and/or chlorphenesin (e.g., both in amounts typically ranging from about 0.5 to about 0.7%, by weight, based on the total weight of the composition).

The preservative system of the present invention makes the use of other preservatives optional. Thus, in certain embodiments the compositions of the present invention are free of any preservative other than the preservative system. Representative examples of other preservatives (which may or may not be present) include alkyl para-hydroxybenzoates, wherein the alkyl radical has from 1, 2, 3, 4, 5 or 6 carbon atoms and preferably from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben), and phenoxyethanol.

### Solvent

The cosmetic compositions of the present invention may contain a solvent. The solvent may be aqueous or non-aqueous in nature, polar or non-polar, or volatile or non-volatile.

The solvent may or may not include water. Typically, the solvent includes water and the cosmetic composition is an emulsion.

Cosmetic compositions that are washable contain water. On the other hand, water may or may not be present in waterproof cosmetic compositions. Generally, the water content of washable cosmetic compositions ranges from about 20 to about 80%, by weight, and in some embodiments from about 30 to about 60%, by weight, based on the total weight of the cosmetic composition. In contrast, the water content of waterproof cosmetic compositions generally ranges from 0 or greater than 0 to about 60%, by weight, and in some embodiments from 0 or greater than 0 to about 35%, by weight, based on the total weight of the cosmetic composition.

One or more water-miscible solvents (having a miscibility in water of greater than 50% by weight at about 25° C.) may also be present. Examples include lower, i.e., C1-C5 monoalcohols such as ethanol and isopropanol, C2-C8 glycols containing such as propylene glycol, ethylene glycol, butylene glycol, dipropylene glycol and pentylene glycol, C₃-C₄ ketones and C₂-C₄ aldehydes. Water-miscible solvents may be present in amounts generally ranging from about 1% to 5%, and in some embodiments, from about 2% to about 4%, by weight, based on the total weight of the composition.

Representative examples of suitable volatile solvents include non-polar volatile hydrocarbon-based oils (which as used herein, refers to oil containing only hydrogen and carbon atoms), silicone oils (optionally comprising alkyl or alkoxy groups that are pendant or at the end of a silicone chain), and fluoro oils. Representative examples of suitable hydrocarbon-based oils include isoparaffins, i.e., branched alkanes containing 8-16 carbon atoms, such as isododecane (also known as 2,2,4,4,6-pentamethylheptane), and petroleum distillates. Representative examples of suitable silicone oils may include linear or cyclic silicones containing from 2 to 7 silicon atoms, and which optionally contain alkyl or alkoxy groups containing from 1 to 10 carbon atoms. Examples include octamethylcyclotetrasiloxane, decamethylcyclopentasiloxane, hexadecamethylcyclohexasiloxane, heptamethylhexyltrisiloxane and heptamethyloctyltrisiloxane.

Representative examples of suitable polar volatile solvents include C₂ to C₅ alcohols, such as ethanol, ethyl 3-ethoxypropionate and isohexyl neopentanoate.

A volatile solvent, if present, is in amounts generally ranging from about 0.1% to about 80%, and in some embodiments, from about 5% to about 70%, and in other embodiments, from about 10% to about 60%, by weight, based on the total weight of the composition.

Representative examples of suitable non-volatile solvents include polyalphaolefins, which include ethylene derivatives oligomerized into even-numbered carbon polyalphaolefins e.g., C₆-C₁₄ olefins such as polydecene and polymers of C₆, C₈, C₁₂ and C14 olefins. The polyolefins may have a molecular weight (MW) generally ranging from about 280 to about 11,500, and a viscosity (CPs at about 20° C.) generally ranging from about 7 to about 32,500. They may also be hydrogenated. In some embodiments, the non-volatile solvent includes PureSyn™ 2 (MW about 283), 4 (MW about 432), 6 (MW about 570), 8 (MW about 611), 150 (MW about 3980) and 300 (MW about 4870) (INCI name: hydrogenated polydecene). The viscosity of these polymers is about 8, about 33, about 64, about 103, about 4179 and about 8400, respectively.) PureSyn™ 100 (MW about 2939, viscosity about 3900, INCI name: hydrogenated C6-14 olefin polymers) and PureSyn™ 1000 (MW about 11,500, viscosity about 32,400, INCI name: polydecene) may also be useful. The PureSyn™ products are commercially available from Exxon Chemicals.

A non-volatile solvent, if present, is in amounts generally ranging from about 0.1% to about 70%, and in some embodiments, about 0.5% to about 40%, and in other embodiments, 1% to about 25%, by weight, based on the total weight of the composition.

### Wax

The cosmetic compositions of the present invention may include a wax. For the purposes of the present invention, the term “wax” means a lipophilic fatty compound that is solid at room temperature (about 25° C.) and atmospheric pressure (760 mmHg, i.e., 105 Pa), which undergoes a reversible solid/liquid change of state and which has a melting point of greater than 30° C. and in some embodiments, greater than about 55° C. up to about 120° C. or even as high as about 200° C. For the purposes of the invention, the waxes are those generally used in cosmetics and dermatology. A variety of waxes may be useful, including waxes of animal origin, waxes of plant origin, waxes of mineral origin and waxes of synthetic origin. Representative examples of suitable waxes of animal origin include beeswaxes, lanolin waxes and Chinese insect waxes. Representative examples of suitable waxes of plant origin include rice waxes, carnauba wax, candelilla wax, ouricurry wax, cork fibre waxes, sugar cane waxes, Japan waxes, sumach wax and cotton wax. Representative examples of suitable waxes of mineral origin include paraffins, microcrystalline waxes, montan waxes and ozokerites. Representative examples of suitable waxes of synthetic origin include polyolefin waxes, e.g., polyethylene waxes, waxes obtained by Fischer-Tropsch synthesis, waxy copolymers and their esters, silicone and fluoro waxes, and Uniclear® from Arizona Chemical. Alternatively, hydrogenated oils of animal or plant origin may be used. Representative examples include hydrogenated jojoba waxes and hydrogenated oils which are obtained by catalytic hydrogenation of fats composed of a C₈-C₃₂ linear or nonlinear fatty chain, hydrogenated sunflower oil, hydrogenated castor oil, hydrogenated copra oil, hydrogenated lanolin and hydrogenated palm oils. In some embodiments, the compositions contain at least two or at least three waxes.

A wax, if present, is in amounts generally ranging from about 0.1% to about 50%, and in some embodiments from about 10% to about 40%, and in yet other embodiments from about 20% to about 25% by weight, based on the total weight of the composition.

### Surfactant/Emulsifier

In those embodiments wherein the cosmetic composition contains water, the compositions of the invention also contain in preferred embodiments one or more surfactants or emulsifiers to aid in the formation and maintenance of an emulsion with other solvents and/or ingredients, such as waxes and oils. Surfactants useful in the present invention include amphoteric/zwitterionic surfactants, nonionic surfactants, cationic surfactants, and anionic surfactants.

Representative examples of suitable amphoteric surfactants include lauryl betaine, lauroamphoglycinate, lauroamphopropylsulfonate, lauroamphopropionate, lauroampho-carboxyglycinate, lauryl sultane, myristamidopropyl betaine, myristyl betaine, myristoamphoglycinate, myristyl propionate, stearoamphoglycinate, stearoamphopropionate, stearoamphopropylsulfonate, stearyl betaine, cocamidoethyl betaine, cocamidopropyl betaine, cocamidopropyl hydroxysultane, cocamidopropyl dimethylamine propionate, cocoamphoglycinate, cocoamphocarboxypropionate, cocoamphocarboxyglycinate, coco-betaine, cocoamphopropionate, and cocoamphopropylsulfonate.

Representative examples of suitable nonionic surfactants include fatty alcohols, such as cetyl alcohol, fatty acids, e.g., C₁₂-C₁₆ fatty acids, such as stearic acid, fatty acid esters, and alkoxylated, particularly ethoxylated, fatty acid esters of polyhydric alcohols such as glycerols and sorbitol, for example, steareth-20, polyoxyethylene monolaurate, polyoxyethylene monooleate, polyoxyethylene monostearate, sorbitan monolaurate, sorbitan trioleate, generally with a degree of ethoxylation of from about 20 to about 85; mono- and di-alkanolamides, such as the N-acyl derivatives of mono- and di-ethanol amines, and polyethoxylated monoalkanolamides; amine oxides, such as cocoamidopropyl dimethylamine oxides, coco bis-2-hydroxyethyl amine oxides and lauryl dimmethylamine oxide; ethoxylated alkanolamides; ethoxylated oils and fats such as ethoxylated lanolins; and ethoxylated alkylphenols, such as Nonoxynol.

Representative examples of suitable anionic surfactants include the following: sodium stearoyl lactylate, the alkali metal, ammonium, or amine salts of alkyl sulfates, alkyl ether sulfates, linear alpha-olefin sulfonates, dialkyl sulfosuccinates, alkylamidosulfosuccinates, and alkyl taurates each having from about C₁₂ to C₁₈ alkyl or alkenyl groups. Particular examples include the salts of lauryl sulfates and lauryl ether sulfates the latter having an average level of ethoxylation of 1-3.

Representative examples of suitable cationic surfactants include quaternium-16, quaternium-26, quaternium-27, quaternium-30, quaternium-33, quaternium-43, quaternium-52, quaternium-53, quaternium-56, quaternium-60, quaternium-61, quaternium-62, quaternium-70, quaternium-71, quaternium-72, quaternium-75, quaternium-76 hydrolyzed collagen, quaternium-77, quaternium-78, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, and quaternium-79 hydrolyzed wheat protein, quaternium-80, quaternium-81, quaternium-82, quaternium-83, quaternium-84.

In embodiments of the present invention the surfactant is present in an amount of generally ranging from about 0.1% to about 25% by weight, in some embodiments from about 0.5% to about 15%, and in some other embodiments from about 1% to about 10% by weight, based on the total weight of the composition.

In embodiments of the present invention that contain a fatty acid emulsifier, it is preferred to use a co-emulsifier or co-surfactant. Co-surfactants are especially preferred in inventive compositions that contain relatively high amounts of water (e.g., greater than about 30% by weight of the cosmetic composition) and/or wax (e.g., greater than about 20% by weight of the cosmetic composition). Representative examples of co-surfactants/co-emulsifiers that may be suitable for use in the present invention typically have HLB values of less than about 20, such as polyethoxylated alcohols (e.g., Steareth-20, HLB of 15.5) and glycerol esters of fatty acids such as glyceryl stearate (HLB of 4). In some embodiments, the HLB of the co-emulsifier ranges from about 12-17 to about 4-8, and in other embodiments from about 14-16 to about 4-5. The amount of co-emulsifier that may be present in the inventive compositions generally ranges from about 1% to about 10%, and in some embodiments from about 3% to about 6%, based on the total weight of the cosmetic composition.

### Thickener

The cosmetic compositions of the present invention may include a thickener. The thickeners include water-soluble thickeners and oil phase thickeners. Representative examples of suitable water-soluble thickeners include polyvinylpyrrolidone (PVP), polyvinyl alcohol, crosslinked acrylates (e.g. Carbopol 982), hydrophobically-modified acrylates (e.g. Carbopol 1382); polyacrylamides such as, for example, the crosslinked copolymers sold under the names Sepigel 305 (CTFA name: polyacrylamide/C13-C14 isoparaffin/Laureth 7) or Simulgel 600 (CTFA name: acrylamide/sodium acryloyldimethyltaurate copolymer/isohexadecane/polysorbate 80) by SEPPIC; 2-acrylamido-2-methylpropanesulphonic acid polymers and copolymers, that are optionally crosslinked and/or neutralized; cellulose derivatives such as hydroxyethylcellulose, sodium carboxymethylcellulose, hydroxypropyl methylcellulose, hydroxypropyl cellulose, ethyl cellulose and hydroxymethyl cellulose; polysaccharides and gums, e.g., natural gums such as acacia gum, xanthan gum (e.g., Rhodicare XC from Rhodia Inc. (Cranbury, N.J.), sclerotium, carrageenan and pectin; polysaccharide resins such as starch and its derivatives, including hydrolyzed corn starch, hyaluronic acid and its salts, clays, and, in particular, montmorillonites, hectorites, bentonites, and laponites, crosslinked polyacrylic acids, such as the “Carbopol” products from the company Goodrich, the polyglyceryl(meth)acrylates polymers sold under the names “Hispagel” or “Lubragel” by the companies Hispano Quimica or Guardian, crosslinked acrylamide polymers and copolymers, such as those sold under the names “PAS 5161” or “Bozepol C” by the company Hoechst, “Sepigel 305” by the company SEPPIC, crosslinked methacryloyloxyethyltrimethylammonium chloride homopolymers sold under the name “Salcare SC95” by the company Allied Colloid, and associative polymers and, in particular associative polyurethanes.

Representative examples of suitable oil phase thickeners include hydrophobically modified alkali-soluble emulsions (HASE), which are synthesized from an acid/acrylate copolymer backbone and a monomer that connects the hydrophobic groups as side chains, e.g., Aculyn 28, commercially available from Rohm & Haas, and which is synthesized from acrylic acid, acrylate esters, and a beheneth-20 methacrylate ester; modified clays, such as magnesium aluminum silicate and hectorites modified with an ammonium chloride of a C₁₀ to C₂₂ fatty acid, such as hectorite modified with distearyldimethylammonium chloride, also known as quaternium-18 bentonite, such as the products sold or made under the names Bentone 34 by the company Rheox, Claytone XL, Claytone 34 and Claytone 40 sold or made by the company Southern Clay, the modified clays known under the name quaternium-18 benzalkonium bentonites and sold or made under the names Claytone HT, Claytone GR and Claytone PS by the company Southern Clay, the clays modified with stearyldimethylbenzoylammonium chloride, known as stearalkonium bentonites, such as the products sold or made under the names Claytone APA and Claytone AF by the company Southern Clay, and Baragel 24 sold or made by the company Rheox. Other mineral gelling agents include silica, such as fumed silica. The fumed silica may have a particle size ranging from about 5 nm to 200 nm.

A thickener, if present, is in amounts generally ranging from about 0.1% to about 10%, and in some embodiments, about 0.2% to about 5%, by weight, based on the total weight of the composition.

### Colorant

The inventive compositions may also contain a colorant, e.g., pigment. Pigments may be chosen from the organic and/or mineral pigments known in the art. These pigments may be in the form of pigmentary powder or paste. They may be coated or uncoated.

The pigments may be chosen, for example, from natural (mineral) pigments, organic pigments, and pigments with special effects such as nacres or glitter flakes.

Natural pigments may be chosen from, for example, iron oxides, mica (e.g., mica sericite), chromium oxides, manganese violet, ultramarine blue, titanium dioxide, chromium hydrate and ferric blue.

The organic pigment may be chosen from nitroso, nitro, azo, xanthene, quinoline, anthraquinone, phthalocyanin, metal-complex, isoindolinone, isoindoline, quinacridone, perinone, perylene, diketopyrrolopyrrole, thioindigo, dioxazine, triphenylmethane and quinophthalone compounds.

White or colored organic pigments may be chosen from carmine, carbon black, aniline black, melanin, azo yellow, quinacridone, phthalocyanin blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenolic derivatives as described in patent FR 2 679 771.

Examples that may also be mentioned include pigmentary pastes of organic pigments, such as the product sold by the company Hoechst under the names: Jaune Cosmenyl IOG: Pigment Yellow 3 (CI 11710); Jaune Cosmenyl G: Pigment Yellow 1 (CI 11680); Orange Cosmenyl GR: Pigment Orange 43 (CI-71105); Rouge Cosmenyl R: Pigment Red 4 (CI 12085); Carmine Cosmenyl FB: Pigment Red 5 (CI 12490); Violet Cosmenyl RL: Pigment Violet 23 (CI-51319); Bleu Cosmenyl A2R: Pigment Blue 15.1 (CI-74160); Vert Cosmenyl GG: Pigment Green 7 (CI-74260); Noir Cosmenyl R: Pigment Black 7 (CI-77266).

The pigments may also be in the form of composite pigments as described in patent EP 1 184 426. These composite pigments may be compounds of particles comprising a mineral core, at least one binder for ensuring the binding of the organic pigments to the core, and at least one organic pigment at least partially covering the core.

The organic pigment may also be a lake. The term “lake” means insolubilized dyes adsorbed onto insoluble particles, the assembly thus obtained remaining insoluble during use.

The inorganic substrates onto which the dyes are adsorbed are, for example, alumina, silica, calcium sodium borosilicate or calcium aluminium borosilicate, and aluminium.

Among the dyes, mention may be made of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 1 0 (CI 77 002), D&C Green 3 (CI 42 053), D&C Blue 1 (CI 42 090).

An example of a lake that may be mentioned is the product known under the following name: D&C Red 7 (CI 15 850:1).

The pigment may also be a pigment with special effects. The term “pigments with special effects” means pigments that generally create a non-uniform colored appearance (characterized by a certain shade, a certain vivacity and a certain lightness) that changes as a function of the conditions of observation (light, temperature, observation angles, etc.). They thus contrast with white or colored pigments that afford a standard uniform opaque, semi-transparent or transparent shade.

Several types of pigment with special effects exist: those with a low refractive index, such as fluorescent, photochromic or thermochromic pigments, and those with a high refractive index, such as nacres or glitter flakes.

Examples of pigments with special effects that may be mentioned include nacreous pigments such as white nacreous pigments such as mica coated with titanium or with bismuth oxychloride, colored nacreous pigments such as titanium mica with iron oxides, titanium mica with ferric blue or with chromium oxide, titanium mica with an organic pigment of the abovementioned type, and also nacreous pigments based on bismuth oxychloride. Nacreous pigments that may be mentioned include the Cellini nacres sold by Engelhard (mica-TiO₂-lake), Prestige sold by Eckart (mica-TiO₂), Prestige Bronze sold by Eckart (mica-Fe₂O₃), and Colorona sold by Merck (mica-TiO₂—Fe₂O₃).

In addition to nacres on a mica support, multilayer pigments based on synthetic substrates such as alumina, silica, sodium calcium borosilicate or calcium aluminium borosilicate, and aluminium, may be envisaged.

Mention may also be made of pigments with an interference effect that are not fixed onto a substrate, for instance liquid crystals (Helicones HC from Wacker), holographic interference flakes (Geometric Pigments or Spectra f/x from Spectratek). Pigments with special effects also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

Quantum dots are luminescent semiconductive nanoparticles capable of emitting, under light excitation, irradiation with a wavelength ranging from 400 nm to 700 nm. These nanoparticles are known from the literature. They may be manufactured in particular according to the processes described, for example, in U.S. Pat. No. 6,225,198 or U.S. Pat. No. 5,990,479, in the publications cited therein, and also in the following publications: Dabboussi B. O. et al. “(CdSe)ZnS core-shell quantum dots: synthesis and characterization of a size series of highly luminescent nanocrystallites” Journal of Physical Chemistry B, vol. 101, 1997, pp. 9463-9475 and Peng, Xiaogang et al. “Epitaxial growth of highly luminescent CdSe/CdS core/shell nanocrystals with photostability and electronic accessibility”, Journal of the American Chemical Society, vol. 119, No. 30, pp. 7019-7029 (1997).

The variety of pigments that may be used makes it possible to obtain a wide range of colors, and also particular optical effects such as metallic effects or interference effects.

A colorant, such as a pigment, if present, is in amounts generally ranging from about 0.01% to about 50%, based on the total weight of the composition. In certain embodiments, the colorant is present in an amount of from about 1% to about 45%, by weight, in other embodiments from about 2% to about 20%, by weight, and in yet other embodiments from about 5% to about 10%, by weight, based on the total weight of the composition. In particular embodiments, the colorant is present in an amount of about 35%, by weight, based on the total weight of the composition.

### Humectant

The compositions of the present invention may contain a humectant. Representative examples of suitable humectants include glycerin, diglycerin, triglycerin, polyglycerin, panthenol, ethoxylated and propoxylated glycerols polypropylene glycol, polyethylene glycol, ethylene glycol, diethylene glycol, triethylene glycol, propylene glycol, dipropylene glycol, hexylene glycol, 1,3-butylene glycol and 1,4-butylene glycol. A preferred humectant is glycerin, butylene glycol, and panthenol.

A humectant if present, is in amounts generally ranging from about 0.1% to about 20%, and in some embodiments, about 0.5% to about 10%, and in other embodiments, 1% to about 7%, by weight, based on the total weight of the composition.

### Chelating Agent

The cosmetic compositions of the present invention may further contain a chelating agent. Cosmetically acceptable chelating agents include amines, carboxylic acids, phosphonic acids, polyphosphoric acids, and metal salts thereof. Representative examples of suitable chelating agents include pentasodium penteate, edetic acid, glutamic tetraacetic acid, asparaginic tetraacetic acid, propyl diamine tetraacetic acid, and metal salts of such acids, such as disodium EDTA. A chelating agent, if present, is in amounts generally ranging from about 0.01% to about 5% by weight of the composition.

### Film-Forming Polymer

The inventive compositions may contain a film-forming polymer. The film-forming polymer may be water-soluble or a polymer soluble in an oil phase. Representative examples of suitable polymers include keratin derivatives, such as keratin hydrolysates and sulphonic keratins; anionic, cationic, amphoteric or nonionic derivatives of chitin or chitosan; cellulose derivatives such as hydroxyethylcellulose, hydropropylcellulose, methylcellulose, ethylhydroxyethylcellulose, carboxymethylcellulose, and quaternized derivatives of cellulose; acrylic polymers or copolymers, such as polyacrylates or polymethacrylates (including homo- and co-polymers thereof), such as sodium polymethacrylate; polyvinylpyrrolidones (PVP) and vinyl copolymers, such as allyl stearate/VA copolymer, and VP/eicosene copolymer, methyl vinyl ether-maleic anhydride copolymers, or vinyl acetate-crotonic acid copolymer; polyester, such as adipic acid/diethylene glycol/glycerin crosspolymer; water-dispersible anionic polyesteramide and/or polyester polymers comprising monomers bearing a functional group—SO₃M, in which M represents a hydrogen atom, an ammonium ion NH₄⁺ or a metal ion, such as, for example, an Na⁺, Mg²⁺, Ca²⁺, Cu²⁺, Fe²⁺ or Fe³⁺ ion. Specific examples of the polymers are described in U.S. Pat. Nos. 3,734,874, 4,233,196 and 4,304,901. Polyurethane polymers, especially anionic, cationic, nonionic or amphoteric polyurethanes, acrylic polyurethanes, polyvinylpyrrolidone polyurethanes, polyester polyurethanes, polyether polyurethanes, polyureas, polyurea/polyurethanes, and mixtures thereof; and polymers of natural origin, modified if desired, such as alginates and carragheenates; glycoaminoglycans, hyaluronic acid and its derivatives; shellac, sandarac gum, dammars, elemis and copals, may also be useful. In an embodiment of the present invention the film-forming polymer is sodium polymethacrylate.

A film-forming polymer, if present, is in amounts generally ranging from about 0.1% to about 20% by weight based on the total weight of the composition.

### Anti-Foam Agent

The compositions of the present invention may also contain an anti-foam agent, such as simethicone, which is a fluid composition containing polydimethylsiloxane and silica. An anti-foam agent, if present, is in amounts generally ranging from about 0.1% to about 5% by weight based on the total weight of the composition.

### Preservative Booster

The compositions of the present invention may also include a preservative booster. A representative example of a suitable preservative booster is sodium dehydroacetate. A preservative booster, if present, is in amounts generally ranging from about 0.1% to about 5% by weight based on the total weight of the composition.

The compositions of the present invention may contain at least one further (e.g., cosmetically or dermatologically acceptable) ingredient, including additives and adjuvants, including, for example, moisturizers, gelling activators, fillers, sunscreen agents (e.g., inorganic sunscreen agent, such as titanium dioxide and zinc oxide and organic sunscreen agents, such as octocrylene, ethylhexyl methoxycinnamate, and avobenzone), dispersion enhancing agents, fibers, antioxidants (e.g., BHT, tocopherol), essential oils, fragrances, and cosmetically active agents and dermatological active agents such as, for example, anti-inflammatory agents, vitamins, and trace elements. These ingredients may be soluble or dispersible in whatever phase or phases is/are present in the cosmetic composition (i.e., aqueous and/or fatty phase).

The compositions for application to keratinous tissue may constitute in particular a cosmetic or dermatological composition for protecting, treating, or caring for keratinous tissue, such as hair, including eyelashes and eyebrows, skin, and lips. In certain embodiments, the cosmetic compositions of the present invention are in the form of mascara, a foundation, an eye shadow, an eyeliner, a blusher, a lipstick, a lipgloss, a care base or balm for the lips, or a concealer product. The cosmetic compositions of the present invention may be applied to keratinous tissue by hand. Alternatively, or in conjunction therewith, they may be applied via an applicator such as a sponge, cotton, brush, or a puff of a natural or synthetic material. In addition, the applicator may be attached to or contained within a container that serves as a reservoir for the cosmetic composition.

It has been surprisingly found that compositions according to an embodiment of the present invention have excellent resistance to microbiological contamination without disrupting an emulsion. However, commonly used preservatives can be ineffective in preserving cosmetic compositions containing ingredients that are particularly susceptible to contamination (e.g., arginine). For example, the use of parabens, such as methyl paraben, with amino acids can disrupt the emulsion when their concentration is too high, yet are ineffective at preservation of the amino acid at low concentrations. Furthermore, parabens tend to hydrolyze in the presence of amino acids, particularly at neutral or slightly basic pH, becoming ineffective at preservation. Alternatively, when triethanolamine is used as a pH adjuster or neutralizer there is the risk of production of nitrosamine byproducts, which are feared to be carcinogens. Embodiments of the present invention make it possible to produce compositions containing arginine, which are both microbiologically stable and free of other preservatives, such as parabens and free of triethanolamine.

The following examples are intended to further illustrate the present invention. They are not intended to limit the invention in any way. Unless otherwise indicated, all parts are by weight.

### EXAMPLES

### Example 1

A mascara composition of the present invention is described below.

| Phase | Ingredient | wt. % |
|---|---|---|
| A | Water | 47 |
| A | Phenoxyethanol | 0.5 |
| A | Water-soluble | 2.5 |
| | solvents | |
| A | Sodium | 0.2 |
| | Dehydroacetate | |
| A | Fillers | 2.1 |
| A | Gums | 4.3 |
| A | Arginine | 1.1 |
| B | Waxes | 24 |
| B | Pigment | 7.2 |
| B | Steareth-20 | 5 |
| B | Stearic Acid | 3.6 |
| C | Caprylyl Glycol | 2.5 |
| | TOTAL | 100 |

The mascara composition described above was prepared as follows. The ingredients of Phase A were added to a side kettle one at a time and mixed until uniform and dispersed. During addition, the side kettle was heated to 80-90° C. so that the main kettle (below) is at the same temperature as the side kettle at emulsification.

The ingredients of Phase B were added to a main kettle and heated to 80-90° C. until all waxes were melted. The pigment was added and ground for 1 hour once waxes were melted. The contents of the side kettle were then added to the main kettle and emulsified for 30 minutes. The main kettle was then cooled to 60° C. using sweeps only. At 60° C., the ingredient of Phase C were added and mixed with sweeps only. The main kettle was cooled to 25-30° C.

### Example 2

A mascara composition of the present invention is described below.

| Phase | Ingredient | wt. % |
|---|---|---|
| A | Water | 44.7 |
| A | Phenoxyethanol | 0.5 |
| A | Water-soluble | 3 |
| | solvents | |
| A | Caprylyl Glycol | 1 |
| A | Sodium | 0.2 |
| | Dehydroacetate | |
| A | Fillers | 0.6 |
| A | Gums | 4.3 |
| A | Arginine | 1.1 |
| B | Waxes | 24 |
| B | Pigment | 7 |
| B | Steareth-20 | 5 |
| B | Stearic Acid | 3.6 |
| C | Denatured Alcohol | 5 |
| | TOTAL | 100 |

This mascara composition was prepared as follows. The ingredients of Phase A were added to a side kettle one at a time and mixed until uniform and dispersed. During addition, the side kettle was heated to 80-90° C. so that the main kettle (below) is at the same temperature as the side kettle at emulsification.

The ingredients of Phase B were added to a main kettle and heated to 80-90° C. until all waxes were melted. The pigment was added and ground for 1 hour once waxes were melted. The contents of the side kettle were then added to the main kettle and emulsified for 30 minutes. The main kettle was then cooled to 25-30° C. and Phase C was added and mixed with sweeps only.

### Example 3

A mascara composition of the present invention is described below

| Phase | Ingredient | wt. % |
|---|---|---|
| A | Water | 44.2 |
| A | Phenoxyethanol | 0.5 |
| A | Water-soluble | 2.5 |
| | solvents | |
| A | Caprylyl Glycol | 0.5 |
| A | Pentylene Glycol | 2 |
| A | Sodium | 0.2 |
| | Dehydroacetate | |
| A | Fillers | 2.1 |
| A | Gums | 4.3 |
| A | Arginine | 1.1 |
| B | Waxes | 24 |
| B | Pigment | 7 |
| B | Steareth-20 | 5 |
| B | Stearic Acid | 3.6 |
| C | Denatured Alcohol | 3 |
| | TOTAL | 100 |

This mascara composition was prepared as described in Example 2.

### Example 4

A mascara composition of the present invention is described below.

| Phase | Ingredient | wt. % |
|---|---|---|
| A | Water | 46.7 |
| A | Phenoxyethanol | 0.5 |
| A | Water-soluble | 3 |
| | solvents | |
| A | Caprylyl Glycol | 1 |
| A | Sodium | 0.2 |
| | Dehydroacetate | |
| A | Fillers | 4.9 |
| A | Gums | 2 |
| A | Arginine | 1.1 |
| A | Pigment | 7 |
| B | Waxes | 22 |
| B | Steareth-20 | 5 |
| B | Stearic Acid | 3.6 |
| C | Denatured Alcohol | 3 |
| | TOTAL | 100 |

This mascara composition was prepared as follows. The ingredients of Phase A were added to a main kettle one at a time and mixed until uniform and dispersed. The pigment was added and ground for 1 hour. During addition, the side kettle was heated to 80-90° C. so that the main kettle (below) is at the same temperature as the side kettle at emulsification.

The ingredients of Phase B were added to a side kettle and heated to 80-90° C. until all waxes were melted. The contents of the side kettle were then added to the main kettle and emulsified for 45 minutes. The main kettle was then cooled to 25-30° C. and Phase C was added and mixed with sweeps only.

### Example 5

A mascara composition of the present invention is described below.

| Phase | Ingredient | wt. % |
|---|---|---|
| A | Water | 36.7 |
| A | Phenoxyethanol | 0.5 |
| A | Water-soluble | 3 |
| | solvents | |
| A | Caprylyl Glycol | 1 |
| A | Sodium | 0.2 |
| | Dehydroacetate | |
| A | Fillers | 4.9 |
| A | Gums | 2 |
| A | Arginine | 1.1 |
| A | Pigment | 7 |
| B | Waxes | 22 |
| B | Steareth-20 | 3 |
| B | Stearic Acid | 3.6 |
| B | Glyceryl Stearate | 3 |
| C | Acrylates Copolymer | 9 |
| D | Denatured Alcohol | 3 |
| | TOTAL | 100 |

This mascara composition was prepared as follows. The ingredients of Phase A were added to a main kettle one at a time and mixed until uniform and dispersed. The pigment was added and ground for 1 hour. During addition, the side kettle was heated to 80-90° C. so that the main kettle (below) is at the same temperature as the side kettle at emulsification.

The ingredients of Phase B were added to a side kettle and heated to 80-90° C. until all waxes were melted. The contents of the side kettle were then added to the main kettle and emulsified for 45 minutes. The main kettle was then cooled to 40° C. and Phase C was added and mixed with sweeps only. The main kettle was then cooled to 25-30° C. and Phase D was added and mixed with sweeps only.

All publications cited in the specification, both patent publications and non-patent publications, are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A cosmetic composition comprising:
a) arginine; and
b) a preservative system comprising from about 0.5% to about 15% of an alcohol, by weight, based on the total weight of the cosmetic composition.

2. The cosmetic composition of claim 1, wherein the preservative system comprises a glycol, ethanol, or a combination of the glycol and ethanol.

3. The cosmetic composition of claim 2, wherein the glycol is present in an amount of 0.5% to about 2.5%, by weight, based on the total weight of the composition.

4. The cosmetic composition of claim 2, wherein the glycol is present in an amount of 0.5% to about 1.5%, by weight, based on the total weight of the composition.

5. The cosmetic composition of claim 2, wherein the glycol is present in an amount of 0.5% to about 1.0%, by weight, based on the total weight of the composition.

6. The cosmetic composition of claim 2, wherein the ethanol is present in an amount of 1.5% to about 10.0%, by weight, based on the total weight of the composition.

7. The cosmetic composition of claim 2, wherein the ethanol is present in an amount of 2.0% to about 6.0%, by weight, based on the total weight of the composition.

8. The cosmetic composition of claim 2, wherein the ethanol is present in an amount of 2.5% to about 3.5%, by weight, based on the total weight of the composition.

9. The cosmetic composition of claim 3, wherein the preservative system comprises caprylyl glycol and ethanol.

10. The cosmetic composition of claim 9, wherein the caprylyl glycol is present in an amount of about 0.5% to about 1.0%, and the ethanol is present in an amount of about 2.5% to about 3.5%, by weight, based on the total weight of the composition.

11. The cosmetic composition of claim 2, wherein the preservative system further comprises phenoxyethanol, chlorphenesin or a combination thereof.

12. The cosmetic composition of claim 11, wherein the phenoxyethanol or the chlorphenesin is present in an amount of about 0.5% to about 0.7%, by weight, based on the total weight of the composition.

13. The cosmetic composition of claim 1, further comprising a solvent, a surfactant, a wax, a thickener, and a colorant.

14. The cosmetic composition of claim 13, wherein the solvent is water.

15. The cosmetic composition of claim 13, wherein the surfactant is a C₁₂-C₁₆ fatty acid.

16. The cosmetic composition of claim 15, wherein the surfactant is stearic acid.

17. The cosmetic composition of claim 15, further comprising a co-surfactant.

18. The cosmetic composition of claim 17, wherein the co-surfactant is a polyethoxylated alcohol.

19. The cosmetic composition of claim 17, wherein the co-surfactant is Steareth-20.

20. The cosmetic composition of claim 17, wherein the co-surfactant is glyceryl stearate.

21. The cosmetic composition of claim 13, wherein the colorant includes black iron oxide.

22. The cosmetic composition of claim 13, further comprising at least one of a humectant, a chelating agent, a film-forming polymer, an anti-foam agent, and a preservative booster.

23. A process for making a cosmetic composition comprising mixing together:
a) arginine; and
b) a preservative system comprising from about 0.5% to about 15% of an alcohol, by weight, based on the total weight of the cosmetic composition.

24. A method of making up or enhancing the appearance of a keratinous tissue comprising applying to the keratinous tissue a cosmetic composition comprising:
a) arginine; and
b) a preservative system comprising from about 0.5% to about 15% of an alcohol, by weight, based on the total weight of the cosmetic composition.
